(19) United States
(12) Patent Application Publication
(10) Pub. No.: US 2020/0136102 A1
(43) Pub. Date: Apr. 30, 2020

(54) SECONDARY BATTERY REMOVABLY ATTACHABLE TO POWER MODULE, AND ELECTRONIC APPARATUS INCLUDING THE SAME

(71) Applicant: Samsung Electronics Co., Ltd., Suwon-si (KR)

(72) Inventors: Kyounghwan Kim, Seoul (KR); Hwiyeol Park, Hwaseong-si (KR); Jeongkuk Shon, Hwaseong-si (KR); Junhyeong Lee, Seoul (KR); Sungjin Lim, Suwon-si (KR); Huisu Jeong, Seongnam-si (KR); Jin S. Heo, Suwon-si (KR)

(21) Appl. No.: 16/561,186

(22) Filed: Sep. 5, 2019

(30) Foreign Application Priority Data
Oct. 25, 2018 (KR) 10-2018-0128407

(51) Int. Cl. H01M 2/06; H01M 2/10; H01M 2/26; H01M 2/30; A61B 5/00

(52) U.S. Cl. CPC H01M 2/06; H01M 2/1022; H01M 2/26; H01M 2/30; A61B 2560/0431; A61B 5/6801; H01M 2220/30; A61B 2560/0214; A61B 5/6846

(57) ABSTRACT

A secondary battery includes a substrate defining first and second through holes; a battery cell on the substrate and including a positive electrode, a separator and a negative electrode; an adhesive layer on the substrate and together with the substrate and the battery cell, removably attachable to an object outside of the secondary battery; a first electrode terminal including a first end electrically connected to the positive electrode and a second end which is opposite to the first end electrically connected to the positive electrode and exposed to the outside of the secondary battery at the first through hole; and a second electrode terminal including a first end electrically connected to the negative electrode and a second end which is opposite to the first end connected to the negative electrode and exposed to the outside of the secondary battery at the second through hole.

### CROSS-REFERENCE TO RELATED APPLICATION

This application claims priority to Korean Patent Application No. 10-2018-0128407, filed on Oct. 25, 2018, and all the benefits accruing therefrom under 35 U.S.C. § 119, the disclosure of which is incorporated herein in its entirety by reference.

### BACKGROUND

### 1. Field

The present disclosure relates to a secondary battery and an electronic apparatus including the same, and more particularly, to a secondary battery which is removably attachable to an object, and an electronic apparatus including the same.

### 2. Description of the Related Art

A secondary battery refers to a battery that is chargeable and dischargeable, unlike a primary battery that cannot be charged. The secondary battery has been widely used in the fields of high-tech electronic devices, including cellular phones, notebook computers, camcorders, etc.

In particular, a lithium secondary battery has a relatively higher voltage and higher energy density per unit weight than a nickel-cadmium battery or a nickel-hydrogen battery. The lithium secondary battery is widely used as a power source of portable electronic equipment, and thus, the demand thereof is increasing.

### SUMMARY

Provided are a secondary battery which is removably attachable to an object, and an electronic apparatus including the same.

Additional features will be set forth in part in the description which follows and, in part, will be apparent from the description, or may be learned by practice of the presented embodiments.

According to an embodiment, a secondary battery includes a substrate defining a first through hole and a second through hole; a battery cell on a first surface of the substrate, the battery cell including a positive electrode, a separator and a negative electrode; an adhesive layer on a second surface of the substrate opposite to the first surface, the adhesive layer together with the substrate and the battery cell being removably attachable to an object outside of the secondary battery; a first electrode terminal including a first end electrically connected to the positive electrode of the battery cell and a second end which is opposite to the first end electrically connected to the positive electrode, the second end of the first electrode terminal being exposed to the outside of the secondary battery at the first through hole of the substrate; and a second electrode terminal including a first end electrically connected to the negative electrode of the battery cell and a second end which is opposite to the first end connected to the negative electrode, the second end of the second electrode terminal being exposed to the outside of the secondary battery at the second through hole of the substrate.

The second end of the first electrode terminal and the second end of the second electrode terminal may be exposed to the outside of the secondary battery at the second surface of the substrate.

The first electrode terminal and the second electrode terminal may be spaced apart from each other within the substrate.

The first electrode terminal or the second electrode terminal may include a protruding portion protruding from the substrate.

The protruding portion may protrude from the second surface of the substrate.

The protruding portion may have a thickness greater than or equal to a thickness of the adhesive layer.

The adhesive layer may be provided on a region of the second surface of the substrate which surrounds the first electrode terminal and the second electrode terminal.

The adhesive layer may be provided on a region of the second surface of the substrate between the first electrode terminal and the second electrode terminal.

The adhesive layer may include a silicone adhesive including polydimethylsiloxane, an acrylic adhesive including polyacrylate, a polyisobutylene-based adhesive, or a combination thereof.

The substrate may include or be formed of a flexible material and insulating material.

The secondary battery may further include a protective layer which seals the battery cell.

The protective layer may include an organic film, an inorganic film, or a combination thereof.

The protective layer may include a metal material covered by a resin layer.

The object may include an electronic apparatus disposed in a living body, an outer surface of the living body, or a covering disposed on the outer surface of the living body.

The battery cell may be provided in plurality and include a first battery cell and a second battery cell which are stacked in a thickness direction of the substrate.

The battery cell may be provided in plurality and include a first battery cell and a second battery cell which are arranged adjacent to each other along the first surface of the substrate.

The secondary battery may further include a first electrode pattern provided on the first surface of the substrate and commonly connected to the positive electrode of each of the first and second battery cells; and a second electrode pattern on the first surface of the substrate and commonly connected to the negative electrode of each of the first and second battery cells. The second end of the first electrode terminal may be in contact with the first electrode pattern, and the second end of the second electrode terminal may be in contact with the second electrode pattern.

According to another embodiment, an electronic apparatus includes the secondary battery described above, and a power receiving module electrically connectable to and disconnectable from the secondary battery, and operable by power supplied thereto from the secondary battery.

The power receiving module may be insertable into a living body.

The power receiving module may be a wearable device or a portable device.

### BRIEF DESCRIPTION OF THE DRAWINGS

These and/or other features will become apparent and more readily appreciated from the following description of the embodiments, taken in conjunction with the accompanying drawings in which:

FIG. 1 is a cross-sectional view illustrating an embodiment of a secondary battery;

FIGS. 2 and 3 are cross-sectional views illustrating other embodiments of secondary batteries;

FIG. 4 is a cross-sectional view illustrating an embodiment of a battery cell;

FIG. 5 is a cross-sectional view illustrating an embodiment of a secondary battery including a plurality of battery cells;

FIGS. 6A is a cross-sectional view and FIG. 6B is a top plan view illustrating another embodiment of a secondary battery including a plurality of battery cells;

FIG. 7 is a cross-sectional view illustrating an embodiment of a secondary battery including a projection-type structure;

FIG. 8A is an enlarged perspective view of a secondary battery and FIG. 8B is a perspective view illustrating an embodiment of a secondary battery attached to an object;

FIG. 8C is a cross-sectional view illustrating an embodiment of a connection between a secondary battery and a power receiving module; and

FIG. 9 is a perspective view illustrating an embodiment of a power receiving module.

### DETAILED DESCRIPTION

Hereinafter, embodiments will be described in detail with reference to the accompanying drawings. In the drawings, the same reference numerals denote the same elements, and the size or thickness of each element may be exaggerated for clarity. When a certain material layer is referred to as being on a substrate or another layer, the material layer may be in direct contact with the substrate or another layer or a third layer may be present between the material layer and the substrate or another layer. In the following embodiments, a material of each layer is merely illustrative and thus other materials may be used. Like reference numerals refer to like elements throughout.

The terminology used herein is for the purpose of describing particular embodiments only and is not intended to be limiting. As used herein, the singular forms “a,” “an,” and “the” are intended to include the plural forms, including “at least one,” unless the content clearly indicates otherwise. “At least one” is not to be construed as limiting “a” or “an.” “Or” means “and/or.” As used herein, the term “and/or” includes any and all combinations of one or more of the associated listed items. Expressions such as “at least one of,” when preceding a list of elements, modify the entire list of elements and do not modify the individual elements of the list.

As used herein, the terms “comprise” and “comprising” should not be construed as necessarily including all of various elements or operations described in the specification, and it should be understood that some of the elements or operations may be omitted or additional elements or operations may be further included.

When an element or layer is referred to as being related to another element such as being “on” or “above” another element or layer, it should be understood to mean that the element or layer is on a top, bottom, a left side, or right side of another element or layer while being in contact with or not in contact with the other element or layer. In contrast, when an element or layer is referred to as being related to another element such as being “directly on” or “above” another element or layer, it should be understood to mean that no intervening layer is present between the element or layer and another element or layer.

The terms first, second, etc. may be used to describe various elements but the elements should not be limited by these terms. The terms are used only for the purpose of distinguishing one element from another element.

Furthermore, relative terms, such as “lower” or “bottom” and “upper” or “top,” may be used herein to describe one element's relationship to another element as illustrated in the Figures. It will be understood that relative terms are intended to encompass different orientations of the device in addition to the orientation depicted in the Figures. For example, if the device in one of the figures is turned over, elements described as being on the “lower” side of other elements would then be oriented on “upper” sides of the other elements. The exemplary term “lower,” can therefore, encompasses both an orientation of “lower” and “upper,” depending on the particular orientation of the figure. Similarly, if the device in one of the figures is turned over, elements described as “below” or “beneath” other elements would then be oriented “above” the other elements. The exemplary terms “below” or “beneath” can, therefore, encompass both an orientation of above and below.

Unless otherwise defined, all terms (including technical and scientific terms) used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

Exemplary embodiments are described herein with reference to cross section illustrations that are schematic illustrations of idealized embodiments. As such, variations from the shapes of the illustrations as a result, for example, of manufacturing techniques and/or tolerances, are to be expected. Thus, embodiments described herein should not be construed as limited to the particular shapes of regions as illustrated herein but are to include deviations in shapes that result, for example, from manufacturing. For example, a region illustrated or described as flat may, typically, have rough and/or nonlinear features. Moreover, sharp angles that are illustrated may be rounded. Thus, the regions illustrated in the figures are schematic in nature and their shapes are not intended to illustrate the precise shape of a region and are not intended to limit the scope of the present claims.

The terms “unit,” “module” or the like described herein refers to a unit for processing at least one function or operation, and may be implemented by hardware, software, a combination thereof.

Hereinafter, embodiments will be described in detail with reference to the accompanying drawings.

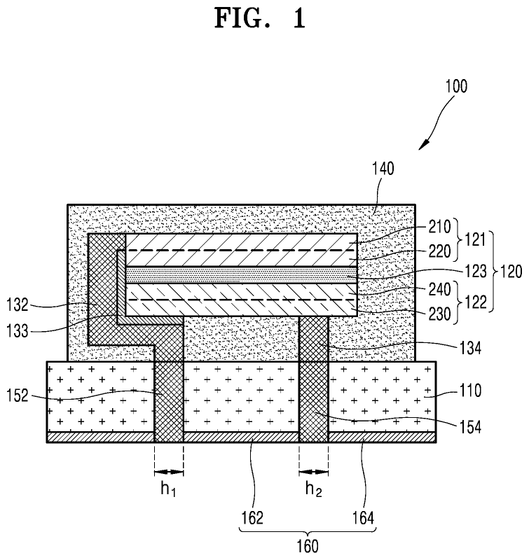

FIG. 1 is a cross-sectional view illustrating an embodiment of a secondary battery 100. As illustrated in FIG. 1, the secondary battery 100 includes a substrate 110 and a battery cell 120 which is provided on a top surface of the substrate 110. The battery cell 120 includes a positive electrode 121, a negative electrode 122, and a separator 123.

The secondary battery 100 and components thereof may be disposed in a plane defined by a first direction and a second direction which cross each other. In FIG. 1, for example, the horizontal direction may represent the first direction and/or the second direction. A thickness of the secondary battery 100 and components thereof may extend along a third direction which crosses each of the first direction and the second direction. In FIG. 1, for example, the vertical direction represents the third direction along which a thickness of the secondary battery 100 and components thereof are defined.

The substrate 110 may include or be formed of a flexible and insulating material. The substrate 110 may include a photo-crosslinkable material, a thermally crosslinkable material and/or a flexible polymeric material. In an embodiment, for example, the substrate 110 may include or be formed of a dielectric material or an insulating material, such as polytetrafluoroethylene (Teflon™), polydimethylsiloxane (“PDMS”), fluorinated ethylene propylene (“FEP”), poly (methyl methacrylate) (“PMMA”), polyvinylidene fluoride (“PVDF”), polycarbonate (“PC”), polyvinyl chloride (“PVC”), polyimide (Kapton®), polypropylene (“PP”), polyethylene (“PE”), polystyrene (“PS”), polyformaldehyde, ethylcellulose, polyamide, melamine formol, perfluoroalkoxy alkane (“PFA”), wool, silk, mica, or nylon, a photoresist such as SU-8, or the like.

The battery cell 120 may be provided on the top surface of the substrate 110 and may include the positive electrode 121 and the negative electrode 122 that are spaced apart from each other, and the separator 123 which is between the positive electrode 121 and the negative electrode 122. Each of the positive electrode 121, the negative electrode 122, and the separator 123 may have a rectangular sheet shape. The negative electrode 122, the separator 123, and the positive electrode 121 may be sequentially stacked on the substrate 110 but embodiments are not limited thereto. The positive electrode 121, the separator 123, and the negative electrode 122 may be sequentially stacked or may be arranged in a different form. In an embodiment, for example, the battery cell 120 may be configured by interposing the separator 123 between the positive electrode 121 and the negative electrode 122, which are in a strip form and shape, and then winding the resultant structure such as about an axis to form a stack of these elements.

The positive electrode 121 may include a positive electrode current collector 210 and a positive electrode active material layer 220 which is on the positive electrode current collector 210. The negative electrode 122 may include a negative electrode current collector 230 and a negative electrode active material layer 240 which is on the negative electrode current collector 230.

The positive electrode current collector 210 may include at least one conductive material such as Cu, Au, Pt, Ag, Zn, Al, Mg, Ti, Fe, Co, Ni, Ge, In, Pd, etc. The positive electrode current collector 210 may include a metal material layer, but is not limited thereto, and may include a layer including of formed of a conductive material other than a metal material.

The positive electrode active material layer 220 may include a Li-containing oxide. The Li-containing oxide may be an oxide including Li and a transition metal. The Li-containing oxide may be, for example, LiMO₂ (here, M=metal). Here, M may be Co, Ni, Mn, or a combination thereof. In an embodiment, the LiMO₂ may be LiCoO₂. The positive electrode active material layer 220 may include a ceramic material having a positive electrode composition and may be a polycrystal or a single crystal. However, the material of the positive electrode active material layer 220 described herein is a merely an example, and other materials may be used.

The negative electrode current collector 230 may include or be formed of, for example, copper foil, nickel foil, stainless steel foil, titanium foil, nickel foam, copper foam, a conductive-metal-coated polymer-based material, or a combination thereof, but is not limited thereto.

The negative electrode active material layer 240 may include or be formed of, but is not particularly limited to, for example, a material for reversibly intercalating or deintercalating lithium ions, a lithium metal, a lithium metal alloy, a material for doping or dedoping lithium, a transition metal oxide, or the like. The material for forming the negative electrode active material layer 240 may further include a binder, a conductive material, and/or a thickener, as well as the material for forming the negative electrode active material layer 240.

The separator 123 separates the positive electrode 121 and the negative electrode 122 from each other and provides a passage for lithium ions therethrough. The separator 123 may include any material suitable for use in lithium batteries. That is, a material having a relatively low resistance to ion movement of an electrolyte and an excellent capability to impregnate the electrolyte may be used. In an embodiment, for example, a material of the separator 123 may be selected from among glass fiber, polyester, PE, PP, polytetrafluoroethylene (“PTFE”) or a combination thereof may be used.

The separator 123 may include nonwoven fabric or woven fabric. In an embodiment, in the lithium ion battery, for example, a separator 123 including a polyolefin-based polymer such as PE or PP is mainly used or a separator 123 which is coated and including a ceramic material or a polymer material may be used to secure heat resistance or mechanical strength. In embodiments, the separator 123 may be provided in a single layer or a multilayer structure.

The battery cell 120 of the secondary battery 100 may include a first lead wire 132 extending from the positive electrode 121, and particularly, from the positive electrode current collector 210 thereof, and a second lead wire 134 extending from the negative electrode 122, and particularly, from the negative electrode current collector 230 thereof. The first lead wire 132 and the second lead wire 134 may respectively include a same material as the positive electrode current collector 210 and the negative electrode current collector 230, respectively, but are not limited thereto. In an embodiment, the first lead wire 132 and the second lead wire 134 may respectively include different materials from the positive electrode current collector 210 and the negative electrode current collector 230.

A first insulating layer 133 may be provided on a region of the first lead wire 132 which is in contact with or faces a region of the battery cell 120 excluding the positive electrode current collector 210 (e.g., at the region of the battery cell 120 including the positive electrode active material layer 220, the negative electrode current collector 230, the negative electrode active material layer 240 and the separator 123). A second insulating layer (not shown) may be provided on a region of the second lead wire 134 which is in contact with or faces a region of the battery cell 120 excluding the negative electrode current collector 230.

In one embodiment, the secondary battery 100 may include a protective layer 140 for sealing the battery cell 120 on the substrate 110 such that no portion of the battery cell 120 is exposed to outside the secondary battery 100 or the battery cell 120 thereof. The protective layer 140 may reduce or effectively prevent deterioration of the battery cell 120 due to air or moisture incident thereto, and reduce or effectively prevent an electrical short circuit from occurring in the battery cell 120.

The protective layer 140 may include an organic material or an inorganic material, and may have a structure in which a layer including or formed of an organic material and a layer including or formed of an inorganic material are alternately stacked. In an embodiment, for example, the protective layer 140 may include, but is not limited to, at least one selected from among polyethylene terephthalate, polybutylene terephthalate, polyethylene naphthalate, polybutylene naphthalate, a polyester copolymer, polycarbonate, a nylon film, polyolefin, and a polyolefin copolymer.

Alternatively, the protective layer 140 may include an outer resin layer, a metal material layer, and an inner resin layer. These layers may be disposed in order, without being limited thereto. The metal material layer within the protective layer 140 may reduce or effectively prevent penetration of moisture, etc. to components of the battery cell 120. The metal material layer within the protective layer 140 may include or be formed of only a metal, or may include or be formed of a resin surrounding metal particles. The metal of the protective layer 140 may include, but is not limited to, at least one material selected from among an alloy of iron (Fe), carbon (C), chromium (Cr) and manganese (Mn), an alloy of iron (Fe), carbon (C), chromium (Cr) and nickel (Ni), copper (Cu), aluminum (Al), and equivalents thereto. Relatively high bending properties or flexibility of the secondary battery 100 may be secured and water barrier properties thereof may be significantly improved by including the metal in the protective layer 140.

In one embodiment, the secondary battery 100 may further include a first electrode terminal 152 and a second electrode terminal 154 which are respectively provided or formed in a first through hole h1 and a second through hole h2 in the substrate 110. The first through hole h1 and the second through hole h2 are spaced apart from each other within the substrate 110 and thus an electrical short circuit between the first electrode terminal 152 and the second electrode terminal 154 may be reduced or effectively prevented. The first electrode terminal 152 and the second electrode terminal 154 are respectively exposed outside of the secondary battery 100, corresponding to positions of the first through hole h1 and the second through hole h2.

A first end of the first electrode terminal 152 may be electrically connected to the positive electrode 121 of the battery cell 120, and a second end of the first electrode terminal 152 opposite to the first end thereof may be exposed to the outside at the first through hole h1. In an embodiment, for example, the first electrode terminal 152 may be electrically connected to the positive electrode 121 via the first lead wire 132. A first end of the second electrode terminal 154 may be electrically connected to the negative electrode 122 of the battery cell 120, and a second end of the second electrode terminal 154 opposite to the first end thereof may be exposed to the outside at the second through hole h2. In an embodiment, for example, the second electrode terminal 154 may be electrically connected to the negative electrode 122 via the second lead wire 134.

The first electrode terminal 152 and the second electrode terminal 154 may include or be formed of a material having relatively high electrical conductivity. In an embodiment, for example, the first electrode terminal 152 and the second electrode terminal 154 may include or be formed of a metal material or the like. In an embodiment, for example, the first electrode terminal 152 and the second electrode terminal 154 may include or be formed of an alloy of iron (Fe), carbon (C), chromium (Cr), and manganese (Mn), an alloy of iron (Fe), carbon (C), chromium (Cr), and nickel (Ni), copper (Cu), aluminum (Al), a metal oxide such as an indium tin oxide (“ITO”) or an indium zinc oxide (“IZO”), a metal (gold (Au) or silver (Ag)) nanoparticle-dispersed thin film, a carbon nanostructure such as carbon nanotube (“CNT”) or graphene, a conductive polymer such as poly 3,4-ethylenedioxythiophene (“PEDOT”), polypyrrole (“PPy”), and poly 3-hexylthiophene (“P3HT”), or the like.

An adhesive layer 160 which is detachably attachable to an object disposed outside the secondary battery 100 may be provided on a bottom surface of the substrate 110. Here, the object to which the adhesive layer 160 is removably attachable may include, but is not limited to, an electronic apparatus within or outside of a living body such as a human, an outer surface of the living body such as human skin, a covering of the living body such as fabric of a garment, or instrument having a certain surface area such as an electronic instrument.

The adhesive layer 160 may be provided at a portion of the bottom surface of the substrate 110. In an embodiment, for example, the adhesive layer 160 may include at least one of a first adhesive layer 162 in a region between the first electrode terminal 152 and the second electrode terminal 154 along the bottom surface of the substrate 110, and a second adhesive layer 164 in remaining regions of the bottom surface of the substrate 110. The adhesive layer 160 may be provided on an entirety of the bottom surface of the substrate 110, excluding a planar area corresponding to the first electrode terminal 152 and the second electrode terminal 154 exposed to outside the secondary battery 100 at the first and second through holes h1 and h2. That is, the adhesive layer 160 is provided on a region of the second surface of the substrate 110 which surrounds the first electrode terminal 152 and the second electrode terminal 154. The adhesive layer 160 may removably fix the secondary battery 100 onto an object and may removably fix the first electrode terminal 152 and the second electrode terminal 154 to be kept in electric contact with electrode terminals of an external device (not shown). The adhesive layer 160 together with the substrate 110 and the battery cell 120 are removably attachable to an object outside of the secondary battery 100.

The adhesive layer 160 may include, but is not limited to, a silicone-based adhesive containing polydimethylsiloxane, an acrylic adhesive containing polyacrylate, a polyisobutylene-based adhesive, or a combination thereof.

The second end of at least one of the first electrode terminal 152 and the second electrode terminal 154 may include a portion protruding downward from the substrate 110 to be disposed outside the substrate 110. The protruding portion may have a same thickness as the adhesive layer 160. Accordingly, no step is formed between a bottom surface of the adhesive layer 160 and a distal end of the second end of a respective one of the first electrode terminal 152 and the second electrode terminal 154, and thus the respective one of the first electrode terminal 152 and the second electrode terminal 154 may be kept in electric contact with electrode terminals of an external device disposed outside of the secondary battery 100 while the adhesive layer 160 is adhered to an object. However, embodiments are not limited thereto, and a thickness of the protruding portion of the first electrode terminal 152 or the second electrode terminal 154 may be greater than a thickness of the adhesive layer 160. Accordingly, the respective one of the first electrode terminal 152 and the second electrode terminal 154 includes the protruding portion, a connection failure between the first electrode terminal 152 and second electrode terminal, and a power receiving module (not shown), may be reduced or effectively prevented. The power receiving module receives power from the secondary battery 100 which is connected thereto.

Since the battery cell 120 is fixed onto the substrate 110 which is flexible, and the adhesive layer 160 is provided on the bottom surface of the substrate 110, the battery cell 120 is relatively easily detachable from and attachable to an object outside of the secondary battery 100, such as to removably connect the secondary battery 100 to a power receiving module disposed relative to the object. When the secondary battery 100 which is relatively easily attachable to and detachable from an object is used, an overall size or dimension of a power receiving module may be greatly reduced because a battery included within the power receiving module is obviated. In addition, since a portion of the secondary battery 100 is not inserted into the power receiving module for attachment thereto, charging and/or replacing of the secondary battery 100 is simplified. A secondary battery 100 detached from an object may maintain the adhesive layer 160 within a structure of the secondary battery 100, without being limited thereto.

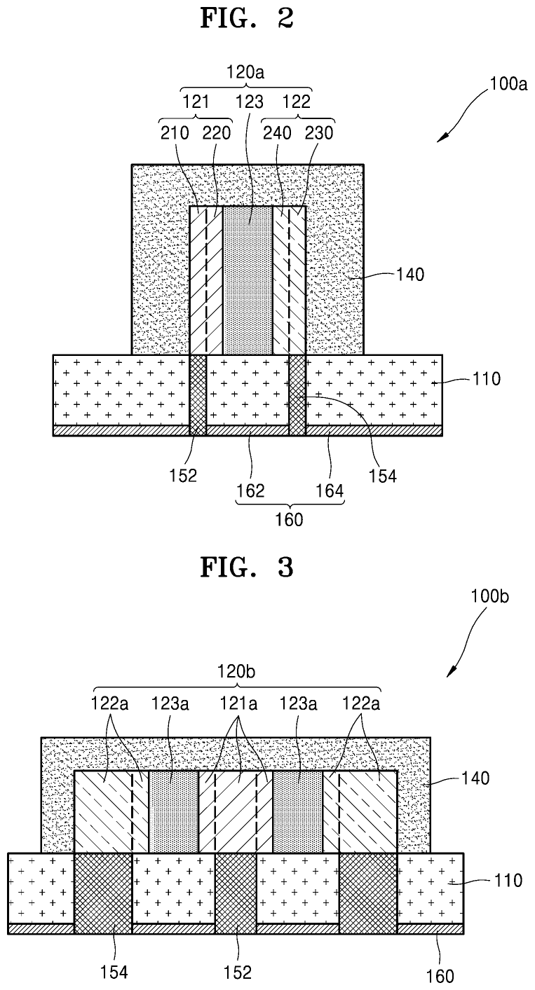

FIGS. 2 and 3 are cross-sectional views illustrating other embodiments of secondary batteries 100a and 100b.

As compared to FIG. 1, the secondary battery 100a of FIG. 2 may include a battery cell 120a in which a positive electrode 121, a separator 123, and a negative electrode 122 are sequentially arranged along a direction which is perpendicular to a thickness direction (e.g., vertical in FIG. 2) of a substrate 110. A positive electrode current collector (210 in FIG. 1) of the positive electrode 121 may correspond to a first electrode terminal 152 along the thickness direction to be in contact with the first electrode terminal 152, and a negative electrode current collector (230 in FIG. 1) of the negative electrode 122 may correspond to a second electrode terminal 154 along the thickness direction to be in contact with the second electrode terminal 154. Accordingly, a first lead wire (132 in FIG. 1) or a second lead wire (134 in FIG. 1) are obviated for respective connection of the battery cell 120a to each of the first electrode terminal 152 and the second electrode terminal 154, and thus manufacturing of the secondary battery 100a is simplified.

Alternatively, as illustrated in FIG. 3, the secondary battery 100b may include a battery cell 120b as a can-type configuration. In an embodiment, for example, the battery cell 120b includes a positive electrode 121a as a core-type configuration, a negative electrode 122a as a shield-type configuration surrounding an outer side of the positive electrode 121a, and a separator 123a as a shield-type configuration between the positive electrode 121a and the negative electrode 122a. With reference to the dotted lines in FIG. 3, the positive electrode 121a shows a positive electrode material layer (220 in FIG. 1) at opposing sides of a positive electrode current collector (210 in FIG. 1). Portions of the separator 123a in FIG. 3 are disposed at opposing sides of the positive electrode 121a. With reference to the dotted lines in FIG. 3, the negative electrode 122a shows a negative electrode active material layer (240 in FIG. 1) between the separator 123a and a negative electrode current collector (230 in FIG. 1). The positions of the positive electrode 121a and the negative electrode 122a may be changed from those illustrated in FIG. 3.

The first electrode terminal 152 and the second electrode terminal 154 may be provided extended through a thickness of the substrate 110 to respectively correspond to a planar shape of the positive electrode 121a (particularly, of the positive electrode current collector, 210 in FIG. 1) and a planar shape of the negative electrode 122a (particularly, of the negative electrode current collector, 230 in FIG. 1) but are not limited thereto. The first electrode terminal 152 and the second electrode terminal 154 may be provided to correspond to some regions of the positive electrode current collector and the negative electrode current collector within FIG. 3, among all portions thereof. As shown in FIG. 3, for example, the second electrode terminal 154 may be electrically connected to one region of the negative electrode current collector among all the regions thereof.

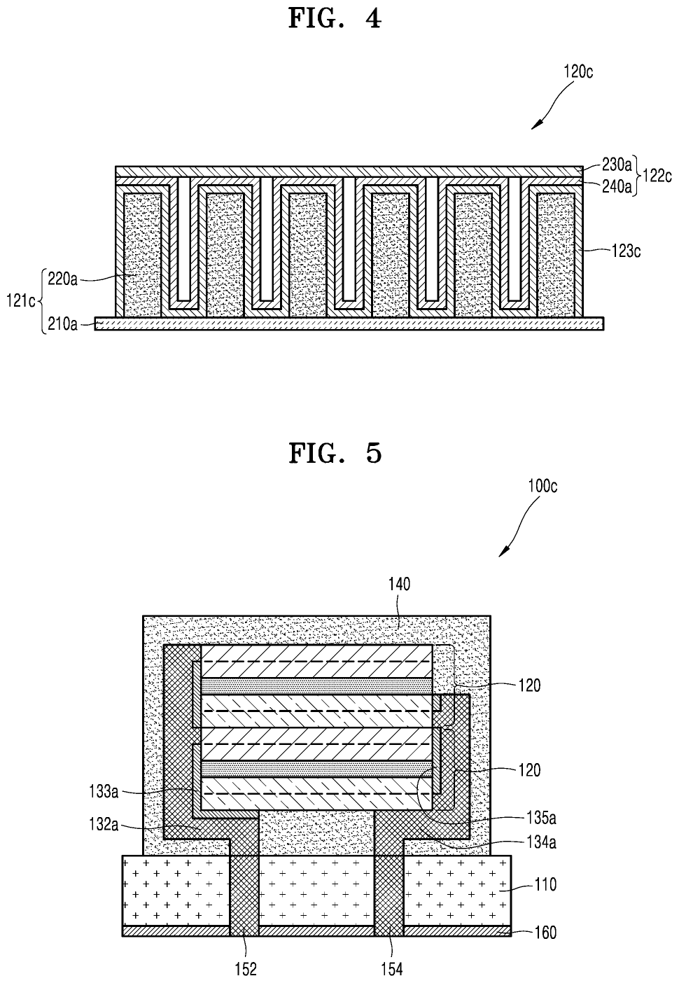

FIG. 4 is a cross-sectional view illustrating another embodiment of a battery cell 120c. The battery cell 120c of FIG. 4 may be used in one or more of the previously described secondary battery configurations shown in FIGS. 1 to 3, without being limited thereto.

The battery cell 120c may include a three-dimensional (“3D”) structure. In an embodiment, for example, the battery cell 120c may include a positive electrode current collector 210a as a sheet-type configuration and a negative electrode current collector 230a as a sheet-type configuration which are spaced apart from each other, a positive electrode active material layer 220a provided in plurality arranged along a top surface of the positive electrode current collector 210a and each extended along a thickness direction from the top surface of the positive electrode current collector 210a in a direction normal thereto, a separator 123c provided on each of the plurality of the positive electrode active material layer 220a to be disposed commonly therewith, and a negative electrode active material layer 240a provided on the separator 123c.

For the battery call 120c in FIG. 4, the positive electrode active material layer 220a may include a 3D structure having a relatively high aspect ratio. Here, the aspect ratio refers to a ratio between a width taken along the top surface of the positive electrode current collector 210a and a height taken along the thickness direction of the 3D structure. The positive electrode active material layer 220a may have, for example, an aspect ratio of 1:1 or more. The battery cell 120c includes the 3D structure and thus has a relatively high capacity.

Alternatively, a secondary battery may include a plurality of battery cells. FIG. 5 is a cross-sectional view illustrating an embodiment of a secondary battery 100c including a battery cell 120 provided in plurality.

As illustrated in FIG. 5, the plurality of the battery cell 120 may be sequentially stacked in a thickness direction of a substrate 110. A first lead wire 132a may be in contact with a positive electrode current collector (210 in FIG. 1) of each of the plurality of the battery cell 120 to be electrically connected thereto, and an electrical short circuit between the first lead wire 132a and a region of each of the plurality of the battery cell 120 excluding the positive electrode current collector (210 in FIG. 1), may be reduced or effectively prevented due to an insulating layer 133a provided in plurality. Similarly, a second lead wire 134a may be in contact with a negative electrode current collector (230 in FIG. 1) of each of the plurality of the battery cell 120 to be electrically connected thereto, and an electrical short circuit between the second lead wire 134a and a region of each of the plurality of the battery cell 120 excluding the negative electrode current collector, may be reduced or effectively prevented due to an insulating layer 135a provided in plurality. The battery cell 120 of FIG. 1 is applied to FIG. 5 but embodiments are not limited thereto and various types of the battery cell 120 may be arranged within the secondary battery 100c.

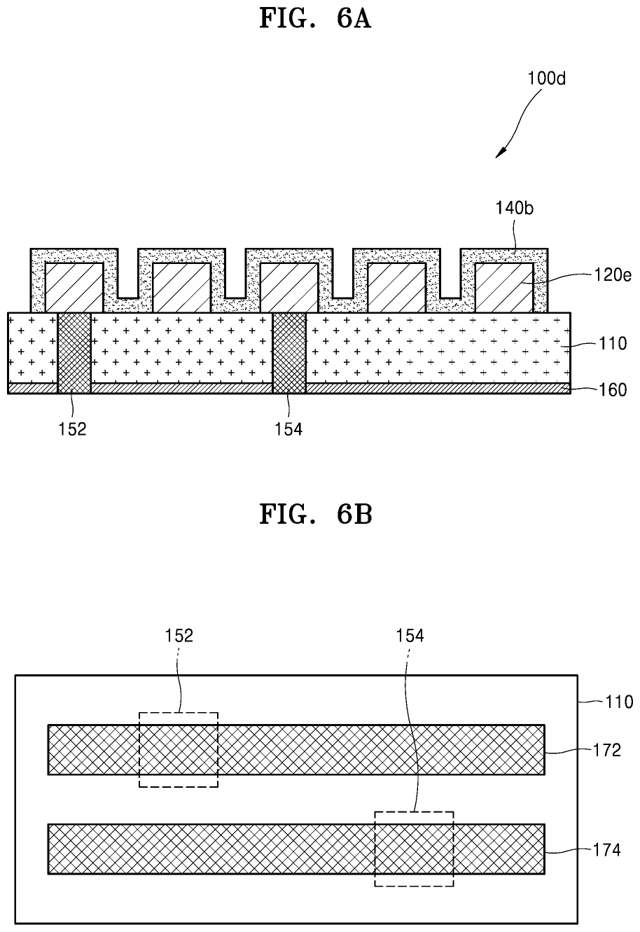

FIG. 6A is a cross-sectional view and FIG. 6B is a top plan view illustrating another embodiment of a secondary battery 100d including a battery cell 120e provided in plurality.

As illustrated in FIG. 6A, the secondary battery 100d may include the battery cell 120e provided in plurality spaced apart from each other. The plurality of the battery cell 120e may have various shapes, e.g., sheet-type battery cells, can-type battery cells, or 3D battery cells such as those previously described herein. A protective layer 140b may be provided on a substrate 110 to cover the plurality of the battery cell 120e.

As illustrated in FIG. 6B, a first electrode pattern 172, which is in contact with a positive electrode current collector (210 in FIG. 1) of each of the plurality of the battery cell 120e, and a second electrode pattern 174, which is in contact with a negative electrode current collector (230 in FIG. 1) of each of the plurality of the battery cell 120e, may be arranged on a top surface of the substrate 110. Where the vertical direction in FIG. 6B represents a first direction, the first electrode pattern 172 and the second electrode pattern 174 are spaced apart from each other in the first direction and may include or be formed of a material having relatively high conductivity. Each of the first electrode pattern 172 and the second electrode pattern 174 are lengthwise extended along a second direction (e.g., horizontal in FIG. 6B) to be commonly connected to the positive electrode current collector (210 in FIG. 1) and the negative electrode current collector (230 in FIG. 1) of each of the plurality of the battery cell 120e. Referring to the dotted line shapes in FIG. 6B, a region of the first electrode pattern 172 may be in contact with a first electrode terminal 152, and a region of the second electrode pattern 174 may be in contact with the second electrode terminal 154. As described above, the secondary battery 100d includes the plurality of the battery cell 120e and thus may have a relatively high capacity.

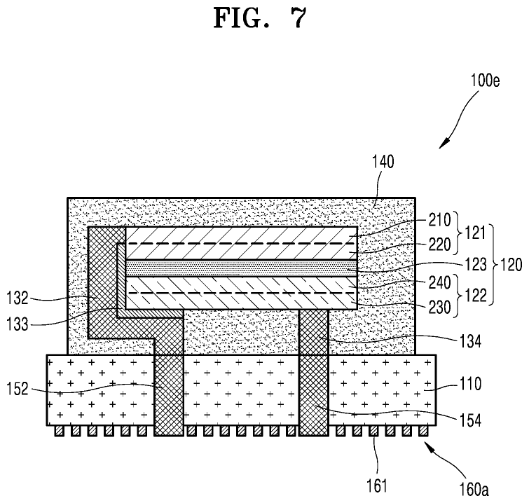

FIG. 7 is a cross-sectional view illustrating an embodiment of a secondary battery 100e including a projection-type structure. As compared to FIG. 1, in the secondary battery 100e of FIG. 7, an adhesive layer 160a provided on a bottom surface of the substrate 110 may have a projection-type structure defined by a protrusion 161 provided in plurality along the bottom surface of the substrate 110. The adhesive layer 160a may have a shape mimicking a gecko animal's bioadhesive system. The secondary battery 100e including the adhesive layer 160a defined by the protrusion 161 provided in plurality may be relatively easily attached to an object (e.g., to the skin of a human) via the adhesive layer 160a and may be easily detached from the object after use. The protrusion 161 provided in plurality may maintain an adhesive function with respect to an object, regardless of the number of times the secondary battery 100e is attached and detached from the object.

A secondary battery in one or more embodiments described above may be a power supply unit of an electronic device which receives power and performs a unique operation with the received power. An electronic device which receives power from the secondary battery may be referred to as a power receiving module. The power receiving module may be a wearable device or a portable device, or may be a medical device that senses biometric information of an object.

Since the secondary battery is separable from the power receiving module, the use and size of the power receiving module may vary. Particularly, when a medical device inserted into a living body such as a human body is a power receiving device, the size of the medical device may be reduced, and the secondary battery may be easily attached to and detached from the medical device while the medical device is inserted into the living body and may be easily charged away from the living body.

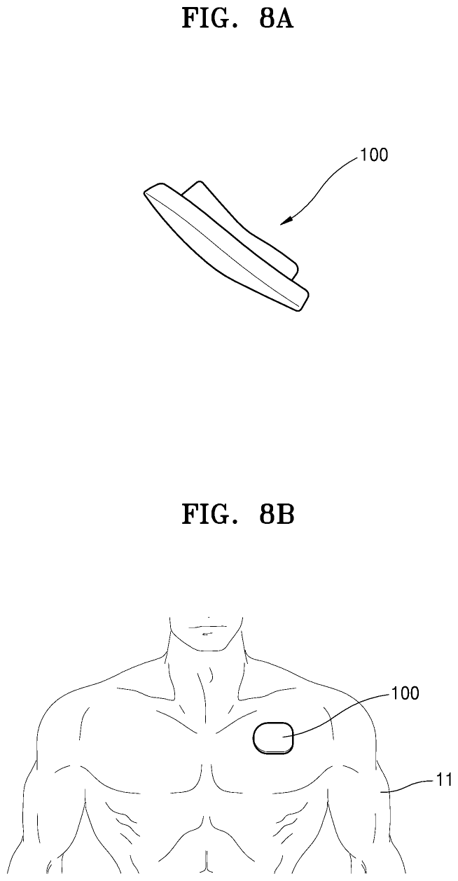

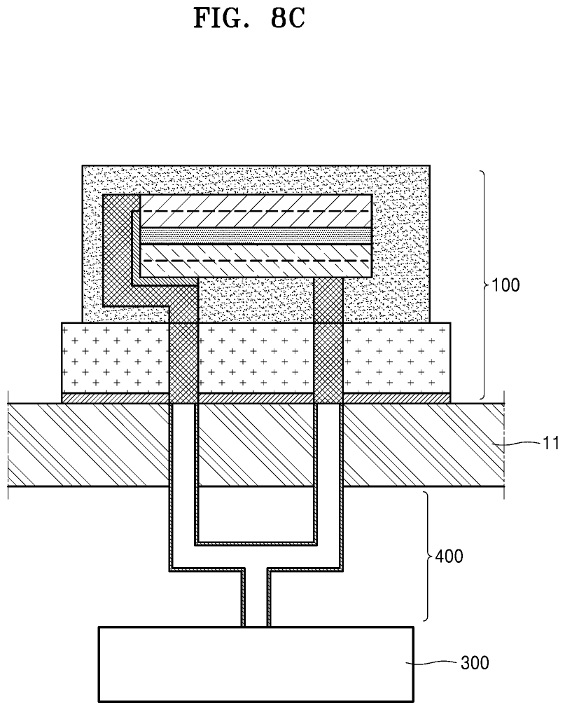

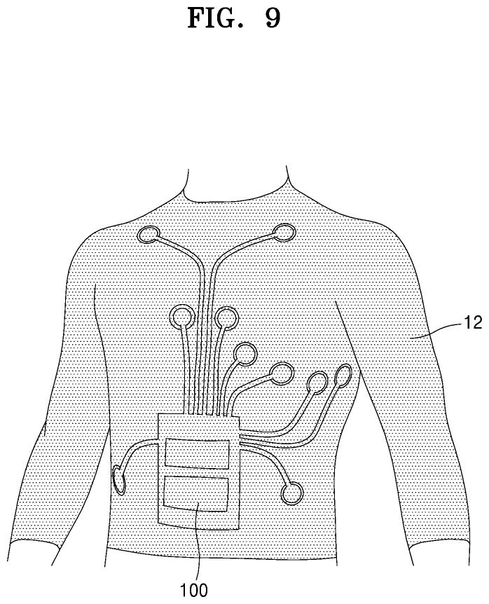

FIG. 8A is an enlarged perspective view of a secondary battery and FIG. 8B is a perspective view illustrating an embodiment of a secondary battery attached to an object. FIG. 8C is a cross-sectional view illustrating an embodiment of a connection between a secondary battery 100 and a power receiving module. In one embodiment, the secondary battery 100 may be electrically connected to the power receiving module which is disposed inside a living body such as a human body, so as to supply power to the power receiving module while disposed inside the human body.

When the power receiving module is inserted into the human body, the secondary battery 100 may be attached to an outer surface of the human body, such as an outer surface defined by human skin 11, as illustrated in FIG. 8A and FIG. 8B. The power receiving module may be a medical device, but is not limited thereto. As illustrated in FIG. 8C, the secondary battery 100 and a power receiving module 300 may be connected to each other by a power line 400. Thus, the secondary battery 100 may supply power to the power receiving module 300 while being attached to the human 18 skin 11, and the power receiving module 300 may perform a unique operation with the supplied power. The power line 400 and the power receiving module 300 may be disposed within the human body. A portion of the power line 400 is connectable to an electrode terminal (152 or 154 in FIG. 1) of the secondary battery 100. The portion of the power line 400 and the electrode terminal of the secondary battery 100 may be connectable to each other at an interface therebetween. The interface may provide a path through which power is transferrable from the secondary battery 100 to the power receiving module 300.

FIG. 9 is a perspective view illustrating an embodiment of a power receiving module 12. As illustrated in FIG. 9, the power receiving module 12 may disposed outside of an object, such as represented by a garment for a living body, where the garment includes a plurality of sensors for sensing biometric information of the living body. The secondary battery 100 may be attachable to the power receiving module 12 to supply power to the plurality of sensors of the garment.

Since a secondary battery and an adhesive layer are arranged on a flexible substrate as described above, the secondary battery may supply power while being adhered to an object. Furthermore, the size of a power receiving module which receives power from the secondary battery may be reduced by using the secondary battery 100 which is relatively easily attachable to and detachable from an object.

In addition, since insertion of a portion of a battery for supplying power into a power receiving module is obviated, stability and ease of battery charging or replacement may be achieved. When the power receiving module is an electronic device configured to be operated while being disposed inside a living body, the secondary battery may be attachable to or detachable from the living body at an outside thereof. Thus, a degree of freedom of selecting a material, size, etc. of the secondary battery may be increased. The secondary battery may be flexible according to a design and/or materials thereof, such that the secondary battery may conform to a shape of an object to which the secondary battery is attached. The secondary battery is removably attachable to the object and thus may be charged or replaced separate from a power receiving module, regardless of a state of the power receiving module.

Although one or more embodiment of a secondary battery is described above with reference to the drawings, it will be understood by those skilled in the art that various modifications may be made therein and equivalent embodiments may be derived therefrom. Accordingly, the embodiments set forth herein should be considered in a descriptive sense only and not for purposes of limitation. The scope of the present disclosure is defined by the appended claims rather than by the foregoing description, and all differences within the scope of equivalents thereof should be construed as being included in the present disclosure.

What is claimed is:

1. A secondary battery comprising:
a substrate defining a first through hole and a second through hole spaced apart from each other;
a battery cell on a first surface of the substrate, the battery cell including a positive electrode, a separator and a negative electrode;
an adhesive layer on a second surface of the substrate opposite to the first surface, the adhesive layer together with the substrate and the battery cell being removably attachable to an object outside of the secondary battery;
a first electrode terminal including a first end electrically connected to the positive electrode of the battery cell and a second end which is opposite to the first end electrically connected to the positive electrode, the second end of the first electrode terminal being exposed to the outside of the secondary battery at the first through hole of the substrate; and
a second electrode terminal including a first end electrically connected to the negative electrode of the battery cell and a second end which is opposite to the first end connected to the negative electrode, the second end of the second electrode terminal being exposed to the outside of the secondary battery at the second through hole of the substrate.

2. The secondary battery of claim 1, wherein the second end of the first electrode terminal and the second end of the second electrode terminal are exposed to the outside of the secondary battery at the second surface of the substrate.

3. The secondary battery of claim 1, wherein the first electrode terminal and the second electrode terminal are spaced apart from each other within the substrate.

4. The secondary battery of claim 1, wherein the first electrode terminal or the second electrode terminal comprises a protruding portion protruding from the substrate to be disposed outside the substrate.

5. The secondary battery of claim 4, wherein the protruding portion protrudes from the second surface of the substrate.

6. The secondary battery of claim 4, wherein a thickness of the protruding portion is greater than or equal to a thickness of the adhesive layer.

7. The secondary battery of claim 1, wherein the adhesive layer is provided on a region of the second surface of the substrate which surrounds the first electrode terminal and the second electrode terminal, and the second end of the first electrode terminal and the second end of the second electrode terminal are each exposed outside the adhesive layer to be exposed to the outside of the secondary battery.

8. The secondary battery of claim 1, wherein
the adhesive layer is provided on a region of the second surface of the substrate which is between the first electrode terminal and the second electrode terminal, and
the second end of the first electrode terminal and the second end of the second electrode terminal are each exposed outside the adhesive layer to be exposed to the outside of the secondary battery.

9. The secondary battery of claim 1, wherein the adhesive layer comprises a silicone adhesive including polydimethylsiloxane, an acrylic adhesive including polyacrylate, a polyisobutylene adhesive, or a combination thereof.

10. The secondary battery of claim 1, wherein the substrate includes a flexible material and insulating material.

11. The secondary battery of claim 1, further comprising a protective layer which seals the battery cell on the substrate.

12. The secondary battery of claim 11, wherein the protective layer comprises an organic film, an inorganic film or a combination thereof.

13. The secondary battery of claim 11, wherein the protective layer comprises a metal material covered by a resin layer.

14. The secondary battery of claim 1, wherein the object comprises an electronic apparatus disposed in a living body, an outer surface of the living body, or a covering disposed on the outer surface of the living body.

15. The secondary battery of claim 1, wherein the battery cell is provided in plurality on the substrate and comprises a first battery cell and a second battery cell which are stacked in a thickness direction of the substrate.

16. The secondary battery of claim 1, wherein the battery cell is provided in plurality on the substrate and comprises a first battery cell and a second battery cell which are arranged adjacent to each other along the first surface of the substrate.

17. The secondary battery of claim 16, further comprising:
a first electrode pattern provided on the first surface of the substrate and commonly connected to the positive electrode of each of the first battery cell and the second battery cell; and
a second electrode pattern on the first surface of the substrate and commonly connected to the negative electrode of each of the first battery cell and the second battery cell,
wherein
the second end of the first electrode terminal is in contact with the first electrode pattern, and
the second end of the second electrode terminal is in contact with the second electrode pattern.

18. An electronic apparatus comprising:
the secondary battery of claim 1; and
a power receiving module electrically connectable to and disconnectable from the secondary battery, and operable by power supplied thereto from the secondary battery.

19. The electronic apparatus of claim 18, wherein the power receiving module is insertable into a living body.

20. The electronic apparatus of claim 18, wherein the power receiving module includes a wearable device or a portable device.
